(19) United States
(12) Patent Application Publication
(10) Pub. No.: US 2017/0206679 A1
(43) Pub. Date: Jul. 20, 2017

(54) SYSTEM AND METHOD FOR THREE-DIMENSIONAL DEPTH IMAGING

(71) Applicant: INRIA INSTITUT NATIONAL DE RECHERCHE EN IFORMATIQUE ET EN AUTOMATIQUE, LE CHESNAY (FR)

(72) Inventors: JULIEN PANSIOT, GRENOBLE (FR); EDMOND BOYER, GRENOBLE (FR); LIONEL REVERET, LA BUISSE (FR)

(73) Assignee: INRIA INSTITUT NATIONAL DE RECHERCHE EN INFORMATIQUE ET EN AUTOMATIQUE, LE CHESNAY (FR)

(21) Appl. No.: 15/324,620

(22) PCT Filed: Jul. 6, 2015
(86) PCT No.: PCT/FR2015/051861

§ 371 Date: Jan. 6, 2017

(30) Foreign Application Priority Data
Jul. 7, 2014 (FR) 1456536

(51) Int. Cl. G06T 11/00; A61B 5/11; A61B 8/00; H04N 13/02; A61B 6/00; G06T 7/00; G06T 7/70; G06T 7/11; A61B 5/00; A61B 8/08

(52) U.S. Cl. CPC G06T 11/003; A61B 5/0035; A61B 5/1128; A61B 5/4528; A61B 8/4416; A61B 8/466; A61B 8/483; A61B 8/5261; A61B 6/4021; G06T 7/0012; G06T 7/70; G06T 7/11; H04N 13/0246; H04N 13/0242; A61B 2505/09; A61B 2576/00; G06T 2207/10081

(57) ABSTRACT

The invention relates to an imaging system and method aimed at constructing a three-dimensional depth image of a patient, which is particularly useful in the field of medical imaging, in particular in the field of X-ray imaging of moving patients. The system includes first imaging means 18 comprising at least one stationary surface-imaging device 3 allowing the acquisition of a sequence of two-dimensional surface images 4 of a patient 2, and a computer processor including a first reconstruction module 5 for constructing a sequence of three-dimensional surface representations 6 of a patient 2 from a series of simultaneous two-dimensional surface images taken in each sequence of two-dimensional surface images 6 acquired by the first imaging means. Second imaging means 19 comprise at least one stationary depth-imaging device 7 allowing the acquisition of a sequence of several two-dimensional depth images 8 of a patient. The computer processor includes a second reconstruction module 9 for constructing a three-dimensional depth representation 1 of the patient from a sequence of three-dimensional surface representations of a patient constructed by the first reconstruction module and a sequence of two-dimensional depth images of the patient acquired by the stationary depth-imaging device.

The present invention relates to a system and a method of imaging aimed at constructing a three-dimensional depth representation of a subject, such as all or part of an object or a body. It notably has an application in the field of medical imaging, in particular in the field of X-raying moving subjects. It may, for example, be applied in the case of analyzing movement in a context of post-operative rehabilitation, and more generally for analyzing the internal dynamics of a patients joints.

Animal or human movement capture, which can be used for a functional analysis of this movement, in recent years has become an increasingly important subject as improvements have been made to acquisition systems.

There are a number of solutions for the capture and analysis of movement in three dimensions, based on the use of visual cues borne by the subject the movement whereof is to be captured. These solutions only allow reconstructing a piece of three-dimensional surface information.

Conversely, X-ray imaging techniques allow the capture of images of the internal structure of a moving subject, but which remain two-dimensional images.

In the field of X-ray imaging, various tomography techniques are known which, by moving an X-ray camera, allow a number of two-dimensional depth images of a subject to be obtained, from which a three-dimensional static depth image may be reconstructed. These techniques all have the drawback of requiring the displacement of the X-ray sensor, the acquisition of a relatively large number of images, and are poorly suited to the acquisition of images of a moving subject.

For example, very expensive computed tomography scanning devices are known, that involve a high dose of radiation and that require total immobility of the subject in a confined environment.

Modified cone-beam tomography devices are also known enabling an isocentric precalibrated movement of the subject, providing more freedom of use and involving a lower dose than with a computed tomography scanner, but which nevertheless still require the immobility of the subject. This is the case, for example, of the method proposed in the publication by J. H. Siewerdsen, D. J. Moseley, S. Burch, S. K. Bisland, A. Bogaards, B. C. Wilson, and D. A. Jaffray, “Volume CT with a flat-panel detector on a mobile, isocentric C-Arm: pre-clinical investigation in guidance of minimally invasive surgery”, Medical physics, 32(1):241-254, 2005.

Also known, from the publication by E. Y. Sidky, C.-M. Kao, and X. Pan, “Accurate image reconstruction from few-views and limited-angle data in divergent-beam CT”, Journal of X-ray Science and Technology, 14(2):119-139, 2006, a method of reconstruction from a calibrated X-ray imaging device, based on the assumption of a limited number of angles of view and/or exposures.

Finally, biplanar beam devices are known for capturing movement from two different points of view. The number of views thus being very limited, these devices generally require a priori models and/or manual intervention, and are limited to the three-dimensional reconstruction of a few characteristic points by simple triangulation.

None of the known X-ray imaging systems or methods allows the three-dimensional and depth reconstruction of a moving subject to be generated, while limiting the dose of radioactivity undergone by the subject.

One of the aims of the invention is therefore notably to resolve the aforementioned problems. Thus, the invention notably has the objective of providing a system and a method for the reconstruction of three-dimensional images of a moving subject, which is inexpensive, and which limits the dose of radioactivity undergone by the subject when the X-ray imaging technique is used.

Thus, the subject matter of the invention, according to a first aspect, is an imaging system intended to construct a three-dimensional depth representation of a subject, such as all or part of an object or a body, including first imaging means comprising at least one fixed surface-imaging device capable of acquiring a sequence of multiple two-dimensional surface images of a subject, and a computer processing unit including a first reconstruction module capable of constructing a sequence of three-dimensional surface representations of a subject from a series of simultaneous two-dimensional surface images taken in each sequence of two-dimensional surface images acquired by the first imaging means.

The device also includes second imaging means comprising at least one fixed depth-imaging device capable of acquiring a sequence of multiple two-dimensional depth images of a subject.

The computer processing unit includes a second reconstruction module capable of constructing a three-dimensional depth representation of the subject from a sequence of three-dimensional surface representations of the subject, constructed by the first reconstruction module and a sequence of two-dimensional depth images of the subject acquired by the fixed depth-imaging device.

According to certain embodiments, the system further includes one or more of the following features, taken in isolation or according to all the technically possible combinations:
the second reconstruction module includes an initial pose determination submodule capable of determining, for each three-dimensional surface representation, an initial pose, defining the position of each point of said three-dimensional surface representation with respect to the position of this said point in a reference three-dimensional surface representation, and the second reconstruction module includes a processing submodule capable of reconstructing a three-dimensional depth representation of the subject from the sequence of initial poses obtained by the initial pose determination submodule and the sequence of two-dimensional depth images of the subject obtained by the second imaging means;
the second reconstruction module includes a pose readjustment submodule capable of readjusting each initial pose with a sequence of two-dimensional depth images of the subject obtained by the second imaging means, and generating a readjusted pose, and the processing submodule is capable of reconstructing a three-dimensional depth representation of the subject from a sequence of readjusted poses obtained by the pose readjustment submodule and the sequence of two-dimensional depth images of the subject obtained by the second imaging means;
the first reconstruction module includes a meshing submodule capable of creating a sequence of three-dimensional meshes of the subject from a series of simultaneous two-dimensional surface images taken in each sequence of two-dimensional surface images acquired by the first imaging means, and the second reconstruction module is capable of constructing the three-dimensional depth representation of the subject from a sequence of three-dimensional meshes of the subject constructed by the meshing submodule and the sequence of two-dimensional depth images of the subject obtained by the second imaging means;
the computer processing unit includes a first segmentation module capable of creating a sequence of two-dimensional surface silhouettes of a subject from a sequence of two-dimensional surface images of the subject acquired by the first imaging means, by segmenting each two-dimensional surface image of its background, and the first reconstruction module is capable of constructing the sequence of three-dimensional surface representations of a subject from a series of simultaneous two-dimensional surface silhouettes taken in each sequence of two-dimensional surface images obtained by the first segmentation module;
the computer processing unit includes a second segmentation module capable of creating a sequence of two-dimensional depth silhouettes of a subject from a sequence of two-dimensional depth images of the subject acquired by the fixed depth-imaging device, by segmenting each two-dimensional depth image of its background, and the second reconstruction module is capable of constructing the three-dimensional depth representation of the subject from the sequence of three-dimensional surface representations of the subject constructed by the first reconstruction module and a sequence of two-dimensional depth images of the subject;
the surface-imaging device is a color imaging, “time-of-flight” imaging device, or a structured light surface sensor, and the depth-imaging device is an X-ray or ultrasound imaging device.

The subject matter of the invention, according to a second aspect, is also an imaging method intended to construct a three-dimensional depth representation of a subject, such as all or part of an object or a body, including a first step of acquiring at least one sequence of multiple two-dimensional surface images of the subject by the first imaging means comprising at least one fixed surface-imaging device, a first step of reconstructing, by a first reconstruction module of a computer processing unit, at least one sequence of three-dimensional surface representations of the subject from a series of simultaneous two-dimensional surface images taken in each sequence of two-dimensional surface images acquired by the first acquisition step.

The method also includes a second step of acquiring at least one sequence of multiple two-dimensional depth images of the subject, by second imaging means comprising at least one fixed depth-imaging device, and a second step of reconstructing, by a second reconstruction module of the computer processing unit, a three-dimensional depth representation of the subject from the sequence of three-dimensional surface representations of the subject, constructed in the first reconstruction step and the sequence of two-dimensional depth images of the subject acquired by the second acquisition step.

According to certain implementations, the method further includes one or more of the following features, taken in isolation or according to all the technically possible combinations:
the second reconstruction step includes a part of an initial pose determination step, by an initial pose determination submodule of the second reconstruction module, for determining, for each three-dimensional surface representation, an initial pose, defining the position of each point of said three-dimensional surface representation with respect to the position of this said point in a reference three-dimensional surface representation, and a processing step, by a processing submodule of the second reconstruction module, for reconstructing the three-dimensional depth representation of the subject from the sequence of initial poses determined by the initial pose determination step and the sequence of two-dimensional depth images of the subject acquired by the second acquisition step;
the reference three-dimensional surface representation is obtained from an external model, or a combination of all or part of the three-dimensional surface representations of the sequence of three-dimensional surface representations;
the second reconstruction step includes a pose readjustment step, by a readjustment submodule of the second reconstruction module, for readjusting each initial pose with the sequence of two-dimensional depth images of the subject obtained by the second acquisition step, and generating a readjusted pose, and the processing step reconstructs the three-dimensional depth representation of the subject from the sequence of poses readjusted by the pose readjustment step and the sequence of two-dimensional depth images of the subject acquired by the second acquisition step;
the first reconstruction step includes a meshing step, by a meshing submodule of the first reconstruction module, for creating a three-dimensional mesh of the subject from a series of simultaneous two-dimensional surface images taken in each sequence of two-dimensional surface images acquired by the first acquisition step, and the second reconstruction step reconstructs the three-dimensional depth representation of the subject from the three-dimensional mesh sequence of the subject constructed in the meshing step and the sequence of two-dimensional depth images of the subject acquired by the second acquisition step;
the first acquisition step includes a first segmentation step, by a first segmentation module, for creating a sequence of two-dimensional surface silhouettes of the subject from each sequence of two-dimensional surface images of the subject previously acquired, by segmenting said two-dimensional surface images of their backgrounds, and the first reconstruction step reconstructs the sequence of three-dimensional surface representations of the subject from a series of simultaneous two-dimensional surface silhouettes taken in each sequence of two-dimensional surface silhouettes obtained by the first segmentation step;
the second acquisition step includes a second segmentation step, by a second segmentation module, for creating a sequence of two-dimensional depth silhouettes of the subject from the sequence of two-dimensional depth images of the subject acquired by the second acquisition step, by segmenting said two-dimensional depth images of their backgrounds, and the second reconstruction step constructs the three-dimensional depth representation of the subject from the sequence of three-dimensional surface representations of the subject constructed in the first reconstruction step, the sequence of two-dimensional depth images obtained by the second imaging means, and the sequence of two-dimensional depth silhouettes of the subject obtained by the second segmentation step;
prior to the first and second acquisition steps, the fixed surface-imaging and depth-imaging devices are calibrated in a common coordinate system;
the first acquisition step is an acquisition step by color imaging, “time-of-flight” imaging or structured light surface sensor devices, and the second acquisition step is an acquisition step by X-ray or ultrasound devices.

Thus, the simultaneous capture of the movement of the internal structure of the subject, such as the skeleton or a part of the skeleton of a person or an animal, and the external surface of this subject, opens significant possibilities of movement analysis, such as the analysis of movement in the case of post-operative rehabilitation, and more generally the analysis of the internal dynamics of a patient's joints.

The combination of at least one two-dimensional surface-imaging device and at least one two-dimensional depth-imaging device, such as an X-ray or ultrasound imaging device, allows the acquisition of the rigid or otherwise movement of a subject without the use of markers.

The acquisition devices assembly remains static, which eliminates the need to use complex mobile systems that must be controlled extremely finely, and which are expensive.

Furthermore, the number of two-dimensional depth images needed for reconstruction is limited, which greatly reduces the dose of radioactivity undergone by the subject when the X-ray imaging technique is used.

The system and the method of the invention do not use models, such as an anatomical model of the subject, thus eliminating the problems of determination of the model and its adjustment.

Consequently, the system and the method of the invention allow the reconstruction of the three-dimensional depth image of a subject of unknown form.

According to the system and the method of the invention, the possible movement of the subject is not considered as noise, but is instead used for reconstruction.

The features and advantages of the invention will appear on reading the following description given solely by way of a non-restrictive example, with reference to the following accompanying figures:

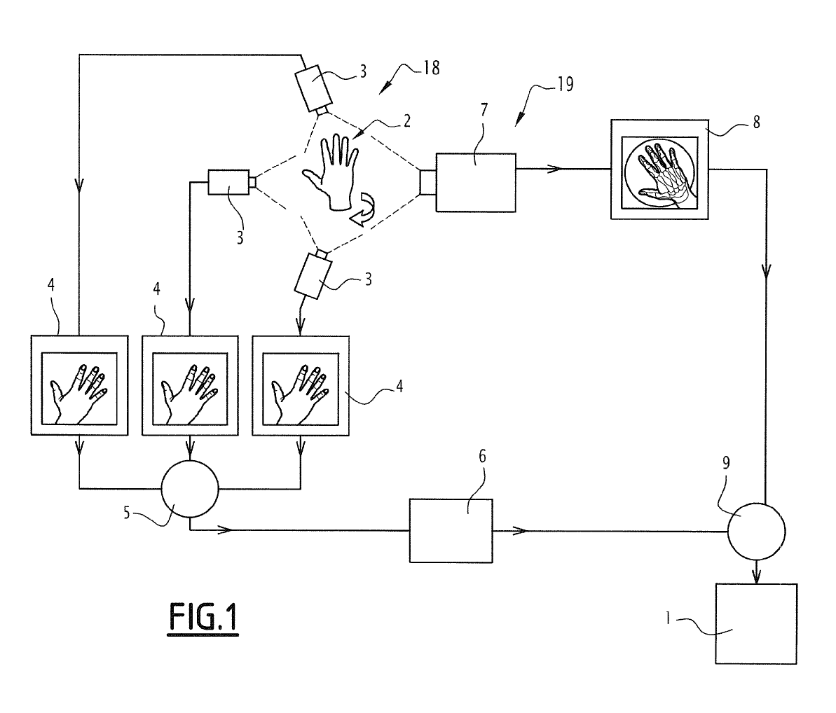

FIG. 1: schematic representation of an example of a system and method according to the invention;

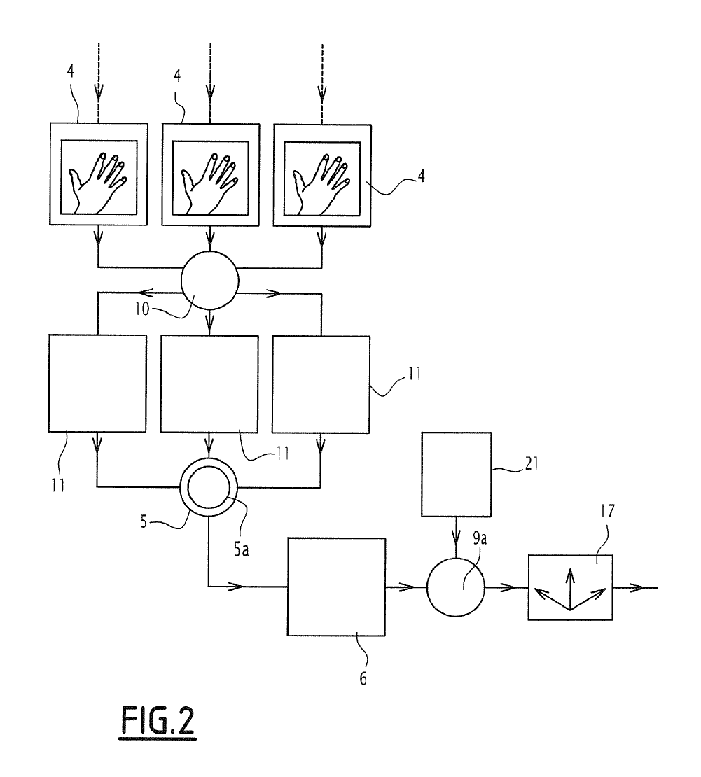

FIG. 2: schematic representation of an embodiment and implementation of a part of the system and the method in FIG. 1 relating to surface acquisition;

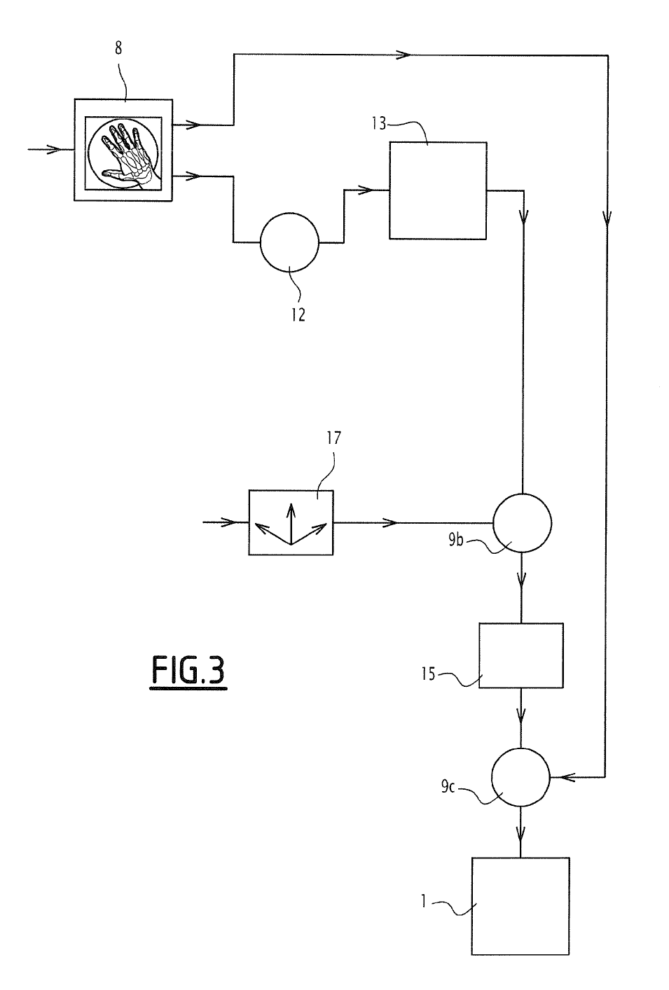

FIG. 3: schematic representation of an embodiment and implementation of the part of the system and the method in FIG. 1 relating to depth acquisition.

The example described with reference to FIGS. 1 through 3 is based on the use of an X-ray image source for the acquisition of information on the internal structure of the subject, combined with a set of color cameras used for constructing the three-dimensional surface representation of the subject, all followed over a given period of time.

The system thus includes first imaging means 18. These imaging means 18 themselves include at least one fixed surface-imaging device 3, such as a color camera 3, a “time-of-flight camera”, or a structured light surface sensor. In the example represented in FIG. 1, the imaging means 18 include three fixed surface-imaging devices 3.

Each camera 3 is used to acquire a sequence of two-dimensional surface images 4 of the subject 2, in this instance a person's hand 2, arranged in the acquisition volume, i.e. the volume observable by the cameras 3.

A computer processing unit, not represented in the figures, allows, by means of a first reconstruction module 5, constructing a series of three-dimensional surface representations 6 of the subject 2, from series of simultaneous, two-dimensional surface images 4, taken in each sequence of two-dimensional surface images 4 acquired by the cameras 3.

Furthermore, the system also includes second imaging means 19. These imaging means 19 include at least one fixed depth-imaging device 7, e.g. an X-ray or ultrasound imaging device.

The depth-imaging device 7 is used to acquire a sequence of two-dimensional depth images 8 of the subject 2.

The computer processing unit further allows, by means of a second reconstruction module 9, constructing a three-dimensional depth representation 1, from a part of a sequence of initial poses 17, in which each initial pose 17 is derived from a three-dimensional surface representation 6, and a sequence of two-dimensional depth images 8 acquired by the fixed depth-imaging device 7.

The concept of initial pose 17 will be explained in more detail with reference to FIG. 2, a little farther on.

The cameras 3 and the fixed depth-imaging device 7 must preferably be calibrated, in a common coordinate system, prior to the acquisition of the images 4, 8.

In one embodiment and implementation, some details of which are represented in FIG. 2, the three-dimensional surface representation 6 takes the form of a three-dimensional mesh 6, created by a meshing submodule 5a of the first reconstruction module 5, from the series of simultaneous two-dimensional surface images 4 taken in each sequence of two-dimensional surface images 4.

Preferably, prior to the implementation of the meshing submodule 5b for obtaining the three-dimensional mesh 6, the two-dimensional surface images 4 are segmented, by means of a first segmentation module 10 of the computer processing unit, so as to create sequences of two-dimensional surface silhouettes 11 that correspond to the segmented two-dimensional surface images 4 of their background.

In a particular embodiment, each two-dimensional surface image 4 is segmented separately from the others. In this case, either the first segmentation module 10 is implemented successively for segmenting each two-dimensional surface image 4, or multiple segmentation modules 10 are implemented in parallel for segmenting multiple two-dimensional surface images 4 simultaneously.

In another particular embodiment, a single segmentation module 10 segments all or part of the two-dimensional surface images 4 at the same time, e.g. by using certain parts of certain of the images for the segmentation of other images.

In a more general way, it is possible to use a combination of the embodiments mentioned above for segmentation, namely a combination of individual and successive segmentations for certain of the two-dimensional surface images 4, individual and parallel segmentation of other two-dimensional surface images 4, and combined and parallel segmentation of yet other two-dimensional surface images 4.

The three-dimensional meshes 6 may be obtained by a polyhedral visual hulls algorithm.

The three-dimensional meshes 6 thus obtained are then compared to a reference mesh 21, or reference three-dimensional surface representation 21, by an initial pose determination submodule 9a of the second reconstruction module 9.

This reference mesh 21 may be, for example, the mesh 6 corresponding to the first series of simultaneous two-dimensional surface images 4 taken in each sequence of two-dimensional surface images 4, and therefore to the first mesh 6 of the sequence of meshes 6.

It may also be the mesh 6 corresponding to any one of the series of simultaneous two-dimensional surface images 4 taken in the sequence of two-dimensional surface images 4, and therefore to any one of the meshes 6 of the sequence of meshes 6.

More generally, this reference mesh 21 may be a combination, such as the average, of all or part of the meshes 6 of the sequence of meshes 6.

Alternatively, this reference mesh 21 may also come from a model external to the system.

More precisely, the initial pose determination submodule 9a uses a robust “iterative closest point”, or ICP, algorithm with detection of aberrations.

This determines how the points of each three-dimensional mesh 6 are positioned, in translation and in rotation, with respect to their position, in translation and rotation, in the reference mesh 21.

Thus, a sequence of initial poses 17 is obtained at the output of the initial pose determination submodule 9a.

As can be seen in FIG. 3, subsequently, a processing submodule 9c of the reconstruction module 9 allows the three-dimensional depth representation 1 of the subject 2 to be reconstructed from the sequence of initial poses 17 obtained by the initial pose determination submodule 9a and the sequence of two-dimensional depth images 8, 13 of the subject 2 obtained by the second imaging means 19.

In one embodiment and implementation, the details of which are represented in FIG. 3, prior to the reconstruction by the processing submodule 9c, of the three-dimensional depth representation 1, a readjustment is performed by a pose readjustment module 9b of the reconstruction module 9, of the two-dimensional depth images 8 and the three-dimensional surface representation 6.

This readjustment generates a sequence of readjusted poses 15, and the processing submodule 9c then reconstructs the three-dimensional depth representation 1 of the subject 2 from the sequence of readjusted poses 15 thus obtained, and the sequence of two-dimensional depth images 8, 13 of the subject 2 obtained by the second imaging means 19.

This readjustment allows the three-dimensional surface representation 6 to be improved, insofar as the three-dimensional meshes 6 include artifacts due to the method and the limited number of cameras 3 used, which generate noise during the creation of this three-dimensional surface representation 6.

For this purpose, the readjustment is preferably implemented not on the two-dimensional depth images 8 but on segmented images 13 of these two-dimensional depth images 8.

Thus, prior to the implementation of a readjustment submodule 12b, the two- dimensional depth images 8 are segmented, by means of a second segmentation module 12, so as to create sequences of two-dimensional depth silhouettes of 13 that correspond to the segmented two-dimensional depth images 8 of their background light.

In the event that multiple fixed depth-imaging devices 7 are used, the considerations regarding the implementation of the first segmentation module 10 described above with reference to FIG. 1, also apply to the implementation of this second segmentation module 12.

The readjustment is based on the assumption that if a three-dimensional representation was perfect, the reprojection of its volume in the plane of the two-dimensional depth image would correspond exactly to the two-dimensional depth silhouette.

A cost function penalizing the differences between the two-dimensional depth silhouettes 13 and the reprojected model is used, with a gradient descent method, for iteratively refining the three-dimensional representation.

This readjustment step also allows for the compensation of a slight spatial and temporal misalignment between the three-dimensional surface reconstruction 6 and the depth silhouettes 13.

For obtaining the three-dimensional depth representation 1, by the reconstruction module 9, in particular by the reconstruction or processing submodule 9c, the method described by S. Kaczmarz in “Angenaherte Auflosung von Systemen linearer Gleichungen”, International Bulletin of the Polish Academy of Science and Letters, Class of Mathematical and Natural Sciences, Series A, Mathematical Sciences, pages 355-357, 1937 (also called the Algebraic Reconstruction Technique or ART) may be used.

This method is therefore used for iteratively reconstructing the three-dimensional depth representation 1.

It is thus necessary that the acquisition of the sequence or sequences of two-dimensional surface images 4 by the first imaging means 18 and the acquisition of the sequence or sequences of two-dimensional depth images 8 by the second imaging means 19, are simultaneous.

Insofar as the quantity and nature of the observed data may make the problem poorly defined locally, a high frequency noise may be found in the result. Consequently, on the assumption that living organisms can be modeled by a relatively homogeneous set of tissues, a three-dimensional adaptation of the method of Rudin et al. “Nonlinear total variation based noise removal algorithms” described in Physica D:Nonlinear Phenomena, 60 (1): 259-268, 1992, is applied to the three-dimensional representation between each iteration.

The present description is given as a non-restrictive example of the invention. Thus, the number of cameras 3 and depth-imaging devices 7, is not restrictive on the invention. Indeed a single depth-imaging device 7 is sufficient for implementing the invention. Also, a single surface-imaging device 3 is sufficient, even if in this case the generation of a three-dimensional surface representation is more complicated. In this case, indeed, the surface-imaging device 3 acquires a sequence of images 4, and each three-dimensional surface representation 6 of the corresponding sequence of three-dimensional surface representations 6, is constructed from a single image 4. To generalize, with N cameras 3, N sequences each including M images 4 are acquired, and a corresponding sequence of M three-dimensional surface images 6 is created, each from N simultaneous images taken in each sequence of M images 4.

In a preferred embodiment, a depth-imaging device 7 and eight surface-imaging devices 3 are used, with 32 images per sequence.

Furthermore, the acquisition technique for the surface images 4 is not necessarily a color imaging technique. Other technologies, such as a “time-of-flight” camera, or a structured light surface sensor, may be used.

Likewise, the acquisition technique for the depth images 8 is not necessarily an X-ray imaging technique. Other techniques, such as ultrasound imaging, may be used.

1. A system effective to construct a three-dimensional depth representation (1) of a subject, including first imaging means comprising at least one fixed surface-imaging device capable of acquiring a sequence of multiple two-dimensional surface images of the subject, and a computer processing unit including a first reconstruction module capable of constructing a sequence of three-dimensional surface representations of the subject from a series of simultaneous two-dimensional surface images taken in each sequence of two-dimensional surface images acquired by the first imaging means, second imaging means comprising at least one fixed depth-imaging device capable of acquiring a sequence of multiple two-dimensional depth images of the subject, and the computer processing unit includes a second reconstruction module capable of constructing the three-dimensional depth representation of the subject from the sequence of three-dimensional surface representations of the subject constructed by the first reconstruction module and a sequence of two-dimensional depth images of the subject acquired by the fixed depth-imaging device.

2. The system as claimed in of claim 1, characterized in that wherein the second reconstruction module includes an initial pose determination submodule configured to determine, for each three-dimensional surface representation, an initial pose, define the position of each point of said three-dimensional surface representation with respect to the position of this said point in a reference three-dimensional surface representation, and in that wherein the second reconstruction module includes a processing submodule configured to construct the three-dimensional depth representation of the subject from the sequence of initial poses obtained by the initial pose determination submodule and the sequence of two-dimensional depth images of the subject obtained by the second imaging means.

3. The system of claim 2, wherein the second reconstruction module includes a pose readjustment submodule configured to readjust each initial pose with a sequence of two-dimensional depth images of the subject obtained by the second imaging means, and generate a readjusted pose, and wherein the processing submodule is configured to reconstruct a three-dimensional depth representation of the subject from a sequence of readjusted poses obtained by the pose readjustment submodule and the sequence of two-dimensional depth images of the subject obtained by the second imaging means.

4. The system of claim 1, wherein the first reconstruction module includes a meshing submodule configured to create a sequence of three-dimensional meshes of the subject from a series of simultaneous two-dimensional surface images taken in each sequence of two-dimensional surface images acquired by the first imaging means, and wherein the second reconstruction module is configured to construct the three-dimensional depth representation of the subject from a sequence of three-dimensional meshes of the subject constructed by the meshing submodule and the sequence of two-dimensional depth images of the subject obtained by the second imaging means.

5. The system of claim 1, wherein the computer processing unit includes a first segmentation module configured to create a sequence of two-dimensional surface silhouettes of a subject from a sequence of two-dimensional surface images of the subject acquired by the first imaging means, by segment of each two-dimensional surface image of its background, and wherein the first reconstruction module is configured to construct the sequence of three-dimensional surface representations of the subject from a series of simultaneous two-dimensional surface silhouettes taken in each sequence of two-dimensional surface images obtained by the first segmentation module.

6. The system of claim 5, wherein the computer processing unit includes a second segmentation module configured to create a sequence of two-dimensional depth silhouettes of a subject from a sequence of two-dimensional depth images of the subject acquired by the fixed depth-imaging device, by segment of each two-dimensional depth image of its background, the second reconstruction module is configured to construct the three-dimensional depth representation of the subject from the sequence of three-dimensional surface representations of the subject constructed by the first reconstruction module and a sequence of two-dimensional depth images of the subject.

7. The system of claim 1, wherein the surface-imaging device is a color imaging, a time-of-flight imaging device, or a structured light surface sensor, and the depth-imaging device is an X-ray or ultrasound imaging device.

8. A method for constructing a three-dimensional depth representation of a subject, including a first step of acquiring at least one sequence of multiple two-dimensional surface images of the subject by the first imaging means comprising at least one fixed surface-imaging device, a first step of reconstructing, by a first reconstruction module of a computer processing unit, at least one sequence of three-dimensional surface representations of the subject from a series of simultaneous two-dimensional surface images taken in each sequence of two-dimensional surface images acquired by the first acquisition step, characterized in that it also wherein the method further includes a second step of acquiring at least one sequence of multiple two-dimensional depth images of the subject, by second imaging means comprising at least one fixed depth-imaging device, and a second step of reconstructing, by a second reconstruction module of the computer processing unit, a three-dimensional depth representation of the subject from the sequence of three-dimensional surface representations of the subject constructed in the first reconstruction step and the sequence of two-dimensional depth images of the subject acquired by the second acquisition step.

9. The method as claimed in claim 8, wherein the second reconstruction step includes a part of an initial pose determination step, by an initial pose determination submodule of the second reconstruction module, for determining, for each three-dimensional surface representation, an initial pose, defining the position of each point of said three-dimensional surface representation with respect to the position of this said point in a reference three-dimensional surface representation, and a processing step, by a processing submodule of the second reconstruction module, for reconstructing the three-dimensional depth representation of the subject from the sequence of initial poses determined by the initial pose determination step and the sequence of two-dimensional depth images of the subject acquired by the second acquisition step.

10. The method as claimed in claim 9, wherein the reference three-dimensional surface representation is obtained from an external model, or a combination of all or part of the three-dimensional surface representations of the sequence of three-dimensional surface representations.

11. The method of claim 9, wherein the second reconstruction step includes a pose readjustment step, by a readjustment submodule of the second reconstruction module, for readjusting each initial pose with the sequence of two-dimensional depth images of the subject obtained by the second acquisition step, and generating a readjusted pose, and wherein the processing step reconstructs the three-dimensional depth representation of the subject from a sequence of poses readjusted by the pose readjustment step and the sequence of two-dimensional depth images of the subject acquired by the second acquisition step.

12. The method of claim 8, wherein the first reconstruction step includes a meshing step, by a meshing submodule of the first reconstruction module, for creating a three-dimensional mesh of the subject from a series of simultaneous two-dimensional surface images taken in each sequence of two-dimensional surface images acquired by the first acquisition step, and wherein the second reconstruction step reconstructs the three-dimensional depth representation of the subject from the three-dimensional mesh sequence of the subject constructed in the meshing step and the sequence of two-dimensional depth images of the subject acquired by the second acquisition step.

13. The method of claim 8 wherein the first acquisition step includes a first segmentation step, by a first segmentation module, for creating a sequence of two-dimensional surface silhouettes of the subject from each sequence of two-dimensional surface images of the subject previously acquired, by segmenting said two-dimensional surface images of their backgrounds, and wherein the first reconstruction step reconstructs the sequence of three-dimensional surface representations of the subject from a series of simultaneous two-dimensional surface silhouettes taken in each sequence of two-dimensional surface silhouettes obtained by the first segmentation step.

14. The method of claim 8, wherein the second acquisition step includes a second segmentation step, by a second segmentation module, for creating a sequence of two-dimensional depth silhouettes of the subject from the sequence of two-dimensional depth images of the subject acquired by the second acquisition step, by segmenting said two-dimensional depth images of their backgrounds, and wherein the second reconstruction step constructs the three-dimensional depth representation of the subject from the sequence of three-dimensional surface representations of the subject constructed in the first reconstruction step, the sequence of two-dimensional depth images obtained by the second imaging means, and the sequence of two-dimensional depth silhouettes of the subject obtained by the second segmentation step.

15. The method of claim 8, wherein prior to the first and second acquisition steps, the fixed surface-imaging and depth-imaging devices are calibrated in a common coordinate system.

16. The method of claim 8, wherein the first acquisition step is an acquisition step by a color imaging device, a time-of-flight imaging device, or a structured light surface sensor device, and the second acquisition step is an acquisition step by X-ray or ultrasound devices.
